(19) United States
(12) Patent Application Publication
(10) Pub. No.: US 2024/0398870 A1
(43) Pub. Date: Dec. 5, 2024

(54) INJECTABLE BIOMATERIAL FOR DYSPHAGIA

(71) Applicant: The Regents of the University of California, Oakland, CA (US)

(72) Inventors: Karen Christman, San Diego, CA (US); Andrew Vahabzadeh-Hagh, La Jolla, CA (US); Emma Zelus, San Diego, CA (US)

(21) Appl. No.: 18/697,375

(22) PCT Filed: Sep. 27, 2022
(86) PCT No.: PCT/US2022/044821

§ 371 Date: Mar. 29, 2024

Related U.S. Application Data
(60) Provisional application No. 63/250,389, filed on Sep. 30, 2021.

(51) Int. Cl. A61K 35/34; A61P 21/00; C12N 5/077

(52) U.S. Cl. CPC A61K 35/34; A61P 21/00; C12N 5/0658; C12N 2509/00

(57) ABSTRACT

Compositions and methods of treatment for head and neck muscle injury or muscle loss, including dysphagia, dysarthria, tongue atrophy, scarring, and fibrosis formation are disclosed herein.

### CROSS-REFERENCE TO RELATED APPLICATIONS

This application claims priority to U.S. Provisional Application No. 63/250,389 filed on Sep. 30, 2021, the entire contents of which are incorporated by reference.

### TECHNICAL FIELD

The present invention relates to compositions and methods of treatment for head and/or neck muscle injury.

### BACKGROUND

Dysphagia is a debilitating condition defined broadly as difficulty swallowing. Dysphagia may result from atrophy, denervation, or fibrosis of the tongue muscles.¹ While this condition is commonly implicated in aging or neurological disorders, another patient population of interest is those recovering from head and neck cancer.² Treatment of these cancers often involves surgical removal of the tumor followed by radiation with or without chemotherapy. About 54,000 cases of oropharyngeal cancer arise annually in the US, with approximately 11,000 deaths.³ Dysphagia is a common sequela of treatment, affecting at least half of head and neck cancer patients.⁴ Dysphagia is a morbid condition that severely affects patient quality of life, and can lead to feeding tube dependence, aspiration, malnutrition, and even death.^{5,6} The current standard of care for oropharyngeal dysphagia is limited to rehabilitative strategies such as swallow therapy and lingual muscle exercises. However, these approaches do not provide long-term improvement in swallowing and tongue strength,⁷ and they do not reverse the tissue-level pathology by improving muscle regeneration and reducing scar tissue formation.⁷

Considering these deficiencies in treatment of tongue dysphagia following head and neck cancer treatment, a number of investigative cell-based therapies that seek to regenerate tongue muscle or to provide structural augmentation to improve tongue function have emerged. While preclinical investigation of allogeneic mesenchymal stem cell injection in an athymic rat model has demonstrated some potential,^{8,9} the manufacturing, cost, and difficulties associated with a living stem cell product pose substantial translational challenges. Additionally, autologous muscle-derived cell therapy demonstrated safety but lacked efficacy after 2 years in a Phase 1 trial.¹⁰ Overall, the current body of research demonstrates a continued need for an accessible and minimally invasive therapeutic that can induce tissue regeneration to reduce scar formation and muscle atrophy to improve muscle repair and restore muscle bulk and function.

### SUMMARY OF THE INVENTION

Compositions and methods of treatment for head and/or neck muscle injury are disclosed herein. In one aspect, a method of treating head and/or neck muscle injury in a subject is provided. This method includes administering to the subject an effective amount of an extracellular matrix composition.

In another aspect, a method of using an extracellular matrix composition is provided. This method includes administering an effective amount of the extracellular composition to the subject. Administering the extracellular matrix composition may be effective to treat head and/or neck muscle injury or muscle loss in the subject.

In yet another aspect, a method of formulating an extracellular matrix composition is provided. This method includes isolating, decellularizing, lyophilizing, milling, and enzymatically digesting a tissue sample. The extracellular matrix composition may be effective to treat head and/or neck muscle injury in a subject.

### BRIEF DESCRIPTION OF THE DRAWINGS

FIG. 1A depicts porcine skeletal muscle chopped into small, uniform pieces for use in formulating skeletal muscle extracellular matrix (SKM) hydrogel.

FIG. 1B depicts the decellularized porcine skeletal muscle of FIG. TA.

FIG. 1C depicts the lyophilized porcine skeletal muscle of FIG. 1B.

FIG. 1D depicts the milled porcine skeletal muscle of FIG. 1C.

FIG. 1E depicts the enzymatically digested porcine skeletal muscle of FIG. 1D.

FIG. 1F depicts SKM hydrogel formulated from porcine skeletal muscle.

FIG. 2 depicts a micrograph of SKM hydrogel post-injection.

FIG. 3A depicts fluorescent analysis of a harvested tongue tissue sample injected with 50 μL SKM hydrogel.

FIG. 3B depicts fluorescent analysis of a harvested tongue tissue sample injected with 100 μL SKM hydrogel.

FIG. 3C depicts fluorescent analysis of a harvested tongue tissue sample injected with 200 μL SKM hydrogel.

FIG. 3D depicts fluorescent analysis of a harvested tongue tissue sample injected with 300 μL SKM hydrogel.

FIG. 4A depicts a brightfield microscopy of a harvested tongue tissue sample injected with 200 μL saline.

FIG. 4B depicts a brightfield microscopy of a harvested tongue tissue sample injected with 200 μL SKM hydrogel.

FIG. 4C depicts a brightfield microscopy of a harvested tongue tissue sample injected with 300 μL SKM hydrogel.

FIG. 4D depicts a box plot comparison of the scar area of the harvested tongue tissue samples of FIGS. 4A-4C.

FIG. 5A depicts fluorescent analysis of a harvested tongue tissue sample injected with 300 μL SKM hydrogel.

FIG. 5B depicts fluorescent analysis of a harvested tongue tissue sample injected with 200 μL saline.

FIG. 5C depicts a high-resolution magnification of the fluorescent analysis of FIG. 5A.

FIG. 5D depicts a high-resolution magnification of the fluorescent analysis of FIG. 5B.

FIG. 5E depicts a box plot comparison of the muscle fiber membrane counts of the harvested tongue tissue samples of FIGS. 5A and 5B.

FIG. 5F depicts a violin plot comparison of the muscle fiber membrane areas of the harvested tongue tissue samples of FIGS. 5A and 5B.

FIG. 5G depicts a box plot comparison of the proportion of muscle fibers with centralized nuclei in the scar area of the harvested tongue tissue samples of FIGS. 5A and 5B.

FIG. 6A depicts a heat map analysis of differentially expressed genes in tongue tissue samples harvested after three days.

FIG. 6B depicts a heat map analysis of differentially expressed genes in tongue tissue samples harvested after seven days.

### DETAILED DESCRIPTION

Various aspects and embodiments of the disclosure are provided by the following description. Before describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. It is also to be understood that the phraseology and terminology employed herein is for the purpose of description and should not be regarded as limiting unless otherwise indicated as such.

Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and uses thereof disclosed herein can be made and executed without undue experimentation in light of the present disclosure.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2nd ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20th ed., (Lippincott, Williams & Wilkins 2003), and Remington, The Science and Practice of Pharmacy, 22th ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the present disclosure described herein include “consisting” and/or “consisting essentially of” aspects and embodiments.

It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

The present disclosure provides decellularized extracellular matrix (ECM) compositions and methods for treatment of head and neck musculature, including the tongue.

For human therapy, there are many source species for the extracellular matrix: e.g., human, porcine, bovine, goat, mouse, rat, rabbit, chicken, and other animal sources. Furthermore, there are many tissue sources for the ECM, e.g., heart, brain, bladder, small intestine, skeletal muscle, kidney, liver, lung, blood vessels and other tissues and organs. In embodiments, the ECM is derived from skeletal muscle.

In embodiments, the injectable skeletal muscle ECM can be prepared through methods such as described in Ungerleider, et al. JACC: Basic to Translational Science, Vol 1, No. 1-2 (2016), which is incorporated by reference in its entirety. In embodiments, the tissue is first decellularized, leaving only the extracellular matrix such as disclosed in U.S. Patent Publication US2013/0251687, for example, which is incorporated by reference in its entirety. The matrix is then lyophilized, ground or pulverized into a fine powder, solubilized with pepsin or other enzymes, and subsequently neutralized and buffered as previously reported. ECM can be lyophilized, and stored in sterile containers. ECM can be resuspended to appropriate/physiological concentration for injection.

In embodiments, a composition having decellularized ECM derived from skeletal muscle tissue or other suitable tissues is provided. The composition may be injectable. The composition may be formulated into a powder or particulate. In embodiments, the composition may be formulated to be in liquid form at room temperature, typically 20° C. to 25° C., and in gel form at a temperature greater than room temperature, or greater than 35° C. In embodiments, the composition may be configured to be delivered to a tissue parenterally, such as through a small gauge needle (e.g., 27 gauge or smaller). In embodiments, the composition may be suitable for direct implantation into a patient. The composition may be formulated either in a dry or hydrated form to be placed on or near injured or missing tissue.

After adjusting concentration, the ECM composition can be lyophilized and stored frozen (e.g. −20C, −80C) for at least 3 months. The ECM composition can then be rehydrated with sterile water prior to injection.

The ECM composition can be directly injected, such as with a 25, 27, or 30 gauge or smaller needle, infused through a catheter, delivered intravenously, or by intravascular infusion with or without a balloon.

The ECM composition gel can be crosslinked with glutaraldehye, formaldehyde, bis-NHS molecules, or other crosslinkers. The ECM composition can be combined with cells, peptides, proteins, DNA, drugs, nutrients, survival promoting additives, proteoglycans, and/or glycosaminolycans. The ECM composition can be combined and/or crosslinked with a synthetic polymer. The ECM composition can be used alone or in combination with above described components for endogenous cell ingrowth, angiogenesis, and regeneration. The ECM composition can be use alone or in combination with above described components as a matrix to change mechanical properties of the tissue. The ECM composition can be delivered with cells alone or in combination with above described components for regenerating damaged tissue.

In embodiments, the present disclosure provides that the ECM is combined with cells, peptides, proteins, DNA, drugs, nanoparticles, antibiotics, growth factors, nutrients, exosomes and extracellular vesicles, survival promoting additives, proteoglycans, and/or glycosaminoglycans.

In embodiments, the present disclosure provides that the composition of infusible extracellular matrix is derived from human, animal, embryonic, and/or fetal tissue sources. In embodiments, the present disclosure provides that the composition of infusible extracellular matrix is derived from heart, brain, bladder, small intestine, skeletal muscle, kidney, liver, lung, bronchioles, blood vessels, and other tissues/organs tissue sources.

In embodiments, the composition is a liquid form of skeletal muscle matrix can assembly into a fibrous scaffold upon injection in vivo. The material can also be processed into a lyophilized form that requires only sterile water, phosphate buffered saline (PBS), or saline to resuspend prior to injection, which can provide ease of storage and use in a clinical setting.

In embodiments, the composition further includes cells, drugs, proteins, or polysaccharides. In embodiments, the composition is delivered as a liquid, and in many instances, the composition may transition to a gel form after delivery. In embodiments, the composition is delivered as a powder.

In embodiments, the composition includes native proteins. In embodiments, the composition includes native peptides. In embodiments, the composition includes native glycosaminioglycans. In embodiments, the composition also includes non-naturally occurring factors that recruit cells into the composition, encourage growth, or prevent infection. In embodiments, the composition including decellularized ECM derived from skeletal muscle tissue retains native glycosaminoglycans. In embodiments, the composition includes naturally occurring factors that recruit cells into the composition, encourage growth, or prevent infection.

In embodiments, the composition further includes a population of exogenous or autologous therapeutic cells. The cells may be stem cells or other precursors of skeletal muscle cells or other cell types.

In embodiments, the composition further includes a therapeutic agent, and as such, is configured as a drug delivery vehicle.

In embodiments, a method of producing a composition with decellularized ECM derived from skeletal muscle or other tissue is provided, the method including the steps of: obtaining from a subject a skeletal muscle or other suitable tissue sample having an extracellular matrix and non-extracellular matrix components; processing skeletal muscle or other tissue sample to remove the non-extracellular matrix component to obtain decellularized skeletal muscle or other tissue extracellular matrix and extracellular proteins and polysaccharides; and sterilizing the decellularized skeletal muscle or other tissue extracellular matrix. In embodiments, the method is performed aseptically without sterilization. In embodiments, the method further includes the step of lyophilizing and grinding up the decellularized ECM. In embodiments, the method further includes the step of enzymatically treating, solubizing, or suspending the decellularized ECM. In embodiments, the decellularized ECM is digested with pepsin at a low pH.

In embodiments, the method further includes the step of suspending and neutralizing the decellularized ECM in a solution. In embodiments, the solution is a PCS or saline solution, which can be injected through a high gauge needled into the desired tissue or organ. In embodiments, the composition is formed into a gel at body temperature. In embodiment, the composition further includes cells, drugs, proteins, or other therapeutic agents that can be delivered within or attached to the composition before, during, or after gelation.

In embodiments, the composition may be used for gel therapy. The composition is neutralized and brought to an appropriate concentration using PBS or saline. In embodiments, the solution can then be injected into the injured tissue or a tissue in need. The needle size may be, without limitation, a 22 gauge, 23 gauge, 24 gauge, 25 gauge, 26 gauge, 27 gauge, 28 gauge, 29 gauge, 30 gauge, or smaller. In embodiments, the needle size through which the solution is injected is a 27 gauge needle.

In embodiments, ECM solution or gel can be injected into the injured tissue or other relevant tissue in need, alone or in combination with above-described components for endogenous cell ingrowth, angiogenesis, and regeneration. In embodiments, the ECM or ECM liquid can be sprayed on or into injured tissue or other relevant tissue in need in need, alone or in combination with above-described components for endogenous cell ingrowth, angiogenesis, and regeneration. In embodiments, the composition can also be used alone or in combination with above-described components as a matrix to change mechanical properties of the skeletal muscle or other relevant tissues and/or to restore muscle mass and function. In embodiments, the composition can be delivered with cells alone or in combination with the above-described components for regenerating muscle mass and function. In embodiments, the composition can be used alone or in combination with above-described components for increasing arteriole and capillary density, as well as recruiting more desired cells for tissue repair and regeneration.

In embodiments, when delivering a composition that comprises the decellularized skeletal muscle ECM and exogenous cells, the cells can be from cell sources for treating certain diseases, such as sources for treating tongue injuries or dysphagia, that include allogenic, xenogenic, or autogenic sources. Accordingly, embryonic stem cells, fetal or adult derived stem cells, induced pluripotent stem cells, skeletal muscle progenitors, fetal and neonatal skeletal muscle cells, mesenchymal cells, parenchymal cells, epithelial cells, endothelial cells, mesothelial cells, fibroblasts, hematopoetic stem cells, bone marrow-derived progenitor cells, skeletal cells, macrophages, adipocytes, and autotransplanted expanded skeletal cells can be delivered by a composition herein. In some instances, cells herein can be cultured ex vivo and in the culture dish environment differentiate either directly to skeletal muscle cells, or to bone marrow cells that can become skeletal muscle cells. The cultured cells are then transplanted into the mammal, either with the composition or in contact with the scaffold and other components. Adult stem cells are yet another species of cell that can be part of a composition herein. Adult stem cells are thought to work by generating other stem cells (for example those appropriate to skeletal muscle) in a new site, or they differentiate directly to a skeletal muscle cells in vivo. They may also differentiate into other lineages after introduction to organs, such as the skeletal muscle. The adult mammal provides sources for adult stem cells in circulating endothelial precursor cells, bone marrow-derived cells, adipose tissue, or cells from a specific organ. It is known that mononuclear cells isolated from bone marrow aspirate differentiate into endothelial cells in vitro and are detected in newly formed blood vessels after intramuscular injection. Thus, use of cells from bone marrow aspirate can yield endothelial cells in vivo as a component of the composition. Other cells which can be employed with the invention are the mesenchymal stem cells administered with activating cytokines. Subpopulations of mesenchymal cells have been shown to differentiate toward skeletal muscle generating cell lines when exposed to cytokines in vitro.

In embodiments, the composition may also include cells, drugs, proteins, or other biological material such as, but not limited to, erythropoietin (EPO), stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), stem cell factor (SCF), platelet-derived growth factor (PDGF), endothelial cell growth supplement (EGGS), colony stimulating factor (CSF), growth differentiation factor (GDF), integrin modulating factor (IMF), calmodulin (CaM), thymidinc kinase (TK), tumor necrosis factor (TNF), growth hormone (GH), bone morphogenic proteins (BMP), matrix metalloproteinase (MMP), tissue inhibitor matrix metalloproteinase (TIMP), interferon, interleukins, cytokines, integrin, collagen, elastin, fibrillins, fibronectin, laminin, glycosaminoglycans, hemonectin, thrombospondin, heparan sulfate, dermantan, chondrotin sulfate (CS), hyaluronic acid (HA), vitronectin, proteoglycans, transferrin, cytotactin, tenascin, and lymphokines.

In embodiments, the composition is injectable. An injectable composition can be, without limitation, a powder, liquid, particles, fragments, gel, or emulsion. The injectable composition can be injected into an injured tissue or organ. The composition can recruit, for example and without limitation, smooth muscle, skeletal muscle, progenitors, and stem cells.

In embodiments, without being bound by theory, ECM is able to be delivered through a catheter or needle for treatment of a malady because ECM is shear-thinning. In embodiments, the catheter or needle can be up to 3 to 5 m in length and have an diameter of 30 g or greater.

In embodiments, an effective amount of ECM delivered to a subject includes a concentration of ECM about 1-20 mg ECM to mL of total product. In embodiments, an effective amount of ECM delivered to a subject includes a concentration of ECM about 2-10 mg ECM to mL of total product. In embodiments, an effective amount of ECM delivered to a patient includes a concentration of ECM about 3-6 mg ECM to mL of total product. In embodiments, an effective amount of ECM delivered to a subject includes a concentration of ECM about 4, 5, or 6 mg ECM to mL of total product.

In embodiments, ECM is delivered in an effective amount to a subject once, or is delivered to a subject multiple times. In embodiments, ECM is delivered to a subject on a schedule or in multiple doses, for example, once a day, once a week, once a month, once a year or more or less frequently.

In embodiments, ECM is delivered at different time courses throughout a subject's disease as is most appropriate, for example, immediately post-injury, infection, or diagnosis, or additionally, about one hour, several hours, one day, one week, one or more months, or one or more years after injury, infection, or diagnosis.

In embodiments, the injection or implantation of said ECM composition repairs damage to the tongue sustained by said subject. In embodiments, the injection or implantation of said composition reduces scar formation in said subject. As used herein, the effective amount can be an amount that reduces muscle atrophy in the area of the injection or implantation or treated tissue of the subject. In embodiments, the effective amount is an amount that increases muscle regeneration. In embodiments, the effective amount is an amount that increases muscle function.

As used herein, “patient” or “subject” means a human or animal subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g., prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

In embodiments, the invention provides methods of preventing or treating head and/or neck muscle injury in a subject, the method comprising administering to the subject an effective amount of an extracellular matrix (ECM) composition. In embodiments, the ECM composition is derived from skeletal muscle. In embodiments, the ECM composition comprises a skeletal muscle extracellular matrix (SKM) hydrogel.

In embodiments, the ECM composition is injected at the site of the head and/or neck muscle injury. In embodiments, the effective amount of the ECM composition comprises a concentration of about 1 mg to about 20 mg of ECM per mL of the ECM composition.

In embodiments, the head and/or neck muscle injury is weakness or asymmetry in at least one facial muscle, tongue weakness, pharyngeal musculature, laryngeal musculature weakness and/or thinning, bulking of the inferior turbinates within the nasal cavity, augmentation around the Eustachian tube for Eustachian tube dysfunction and patulous Eustachian tube, dysphagia, dysarthria, tongue atrophy, scarring, or fibrosis formation. In embodiments, the head and/or neck muscle injury is caused by an oral cancer or a tongue cancer. In embodiments, the effective amount of the ECM composition is effective to promote muscle regeneration, engraftment, growth, volume, bulk, or function.

In embodiments, the invention provides methods of using an extracellular matrix (ECM) composition, the method comprising: administering an effective amount of the ECM composition to a subject; wherein administering the ECM composition is effective to treat head and/or neck muscle injury in the subject. In embodiments, the ECM composition comprises a skeletal muscle extracellular matrix (SKM) hydrogel. In embodiments, the ECM composition is injected at the site of the head and/or neck muscle injury, infused via a catheter, administered intravenously, or administered via intravascular infusion. In embodiments, the effective amount of the ECM composition comprises a concentration of about 1 mg to about 20 mg of ECM per mL of the ECM composition. In embodiments, the effective amount of the ECM composition is administered to the subject once. In embodiments, the effective amount of the ECM composition is administered to the subject periodically at an interval of once per day, once per week, once per month, or once per year. In embodiments, the head and/or neck muscle injury is caused by an oral cancer or a tongue cancer, and wherein administering the effective amount of the ECM composition is effective to promote muscle regeneration, engraftment, growth, volume, bulk, or function.

In embodiments, the invention provides extracellular matrix (ECM) compositions, and methods of formulating such ECM compositions, the method comprising: isolating a tissue sample; decellularizing the tissue sample; lyophilizing the tissue sample; milling the tissue sample; and enzymatically digesting the tissue sample; wherein the ECM composition is effective to treat head and/or neck muscle injury in a subject, as described herein.

### EXAMPLES

It will be understood from the foregoing description that various modifications and changes may be made in the various embodiments of the present disclosure without departing from their true spirit. The description provided herein is intended for the purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications, and equivalents are included within the scope of the presently disclosed inventive concepts as defined herein.

Thus, the examples described herein, which include particular embodiments, will serve to illustrate the practice of the presently disclosed inventive concepts, it being understood that the particulars show are by way of example and for purposes of illustrative discussion of particular embodiments of the presently disclosed inventive concepts only and are presented in the cause of providing what is believed to be a useful and readily understood description of procedures as well as the principles and conceptual aspects of the inventive concepts. Changes may be made in the construction and formulation of the various components and compositions described herein, the methods described herein, or in the steps of the sequence of steps of the methods described herein without departing from the spirit and scope of the presently disclosed inventive concepts.

### Example 1: Assessing SKM Hydrogels as Effective Therapy for Head and/or Neck Muscle Injury Following Head and/or Neck Cancer Treatment

Dysphagia is a life-threatening and morbid sequela following treatment of head and neck cancers. However, clinical approaches to dysphagia are limited primarily to rehabilitative exercises, which lack efficacy and particularly have not been shown to improve tongue strength and swallowing function. Cell therapies under investigation also have not demonstrated significant improvement in tongue function clinically, and pose significant challenges for translation, given difficulties with cost, manufacturing, and logistics of cell therapies. Accordingly, there is a need for cost-effective and easily administered therapeutics that can achieve tissue regeneration to revert the muscle atrophy and fibrosis associated with head and neck cancer treatment in order to improve swallow function.

An alternative approach utilizing an acellular and minimally invasive biomaterial to preserve muscle content and reduce fibrosis of the tongue after injury is provided. In particular, this approach utilizes a decellularized extracellular matrix hydrogel for the treatment of tongue fibrosis in a partial glossectomy injury model.

Skeletal muscle extracellular matrix (SKM) hydrogels have previously demonstrated induction of muscle regeneration, neovascularization, and ECM remodeling in several skeletal muscle conditions, including ischemia reperfusion¹⁷ and birth-related mechanical injuries.¹⁹ It was therefore hypothesized that injection of SKM hydrogel could mitigate scar formation and improve muscle regeneration in an animal model of tongue fibrosis, which was previously developed to model pathologies associated with head and neck cancer treatment.⁹

The use of decellularized extracellular matrix (ECM) hydrogels that induce immune modulation, cellular recruitment and differentiation, neovascularization, and ECM remodeling has been explored as a therapeutic in numerous disease phenotypes.¹¹ The ECM is a complex network of proteins vital for structural support and cell signaling; when a whole tissue is decellularized, the ECM remains as an acellular biomaterial with tissue regenerative properties.¹¹ Additionally, due to their thermoresponsive properties, ECM hydrogels can be delivered minimally invasively via injection, and after exposure to physiologic conditions, the liquid forms a hydrogel comprised of a nanofibrous ECM scaffold.¹² With sufficient decellularization, the acellular xenogeneic ECM hydrogels are biocompatible and have been utilized in large^{3,14} and small animal models¹⁵ as well as a human Phase 1 clinical trial that assessed safety and feasibility for intervention in subacute and chronic myocardial infarction.⁶

A decellularized porcine SKM hydrogel has been previously shown to increase vascularization, enhance the recruitment and differentiation of muscle progenitors, and reduce muscle atrophy and cell death in an ischemic injury model.^{4,5,12} SKM hydrogel has further been shown to prevent skeletal muscle atrophy and mitigate fibrotic degeneration, as well as modulate the immune response after mechanical muscle injury.¹⁰

After investigating the therapeutic efficacy and potential mechanisms of action of SKM hydrogel in a preclinical animal model of tongue fibrosis, it was observed that SKM hydrogel reduces scar formation and improves muscle fiber area in a rat tongue partial glossectomy model. SKM hydrogel was also shown to modulate the immune response and upregulate genes related to angiogenesis. Overall, SKM hydrogel injection is a treatment for tongue fibrosis and a minimally invasive acellular therapeutic for oropharyngeal dysphagia.

For example, injection of SKM hydrogel, when delivered two weeks following a partial glossectomy injury, significantly improved histomorphological properties of tongue tissues. The active phase of muscle regeneration was completed 4 weeks after injection, as demonstrated by the proportion of muscle fibers with centralized nuclei in the site of injury,²⁵ so this time point was used for evaluation of histolomorphological changes in the tongue. At this timepoint, 300 μL SKM hydrogel injection reduced scar formation and increased muscle fiber area within the scar region. These data indicate therapeutic efficacy of SKM hydrogel in promoting constructive remodeling and reversing fibrosis of the tongue consequent to surgical resection of the tissue. Interestingly, a smaller injection volume of SKM hydrogel did not significantly reduce scar area as compared to saline controls. This is likely due to the shorter retention time with the smaller volume, which was insufficient to induce a therapeutic effect and extensive histomorphological changes.

Consistent with previous studies on ECM hydrogels,^{29,26} SKM hydrogel injection downregulated pro-inflammatory genes with a concurrent upregulation of chemokines and cytokines associated with a pro-regenerative response. Additionally, an upregulation of Tbx21, which is commonly used as a marker for Th1 cells, was observed, but without concomitant upregulation of Th1 cytokines (IFN-γ, TNFα, IL-2, IL-12). Tbx21 is, however, also expressed in perisynaptic Schwann cells and given the lack of Th1 cytokines, may instead suggest an improvement in reinnervation of the injured muscle.¹⁸ Genes related to other pro-inflammatory cytokines and chemokines were downregulated in SKM, while pro-regenerative or anti-inflammatory cytokines were upregulated. Il10 and Il33 are particularly associated with muscle repair, M2 macrophages, T_{reg} cell activity, and potentially pro-myogenic activity of fibro-adipogenic progenitor cells.^{28,29}

Additionally, SKM hydrogel injection upregulated genes involved in angiogenesis, which supports previous studies that determined SKM hydrogel induces neovascularization in other skeletal muscle injury models.^{17,21} While significant modulation in gene expression related to myogenesis was not observed, which was expected based on observed histomorphological changes, SKM hydrogel injection did induce upregulation of hyaluronic acid synthesis, which is transiently expressed during hypertrophy,³⁰ and other genes related to myofiber contractile proteins. Changes in myogenesis have been observed at 7 days post-injection of SKM hydrogel in a mechanical birth injury model.¹⁹ In contrast, ECM scaffold implants have demonstrated limited improvements in myogenesis following VML injury.³¹ Thus, while it was thought that SKM hydrogel injection would induce myogenesis based on other injury models, SKM hydrogel injection appears to influence tissue repair through alternate mechanisms in VML-type injury. Additionally, significant changes in ECM remodeling pathways were not observed. Potentially, the early timepoint of transcription analysis, which was intended to capture changes in the immune response may have preceded significant alterations in ECM remodeling, based on previous VML studies.³² Further investigation into transcriptional changes in other pathways, such as fibro-adipogenic progenitor²⁸ or pericyte³³ cell activity—populations that may contribute to fibrogenic or myogenic responses—may further characterize SKM's mechanism of action in this injury model.

One limitation of the partial glossectomy injury model is that while it induces muscle damage and scar formation that mimic the pathologies associated with head and neck cancer treatment, it does not include radiation. Future studies are necessary to include radiation for a potentially more severe injury model to further evaluate the efficacy of SKM hydrogel injection. Additionally, the NanoString multiplex gene expression study involved assaying specific sets of genes related to the pathways mentioned, so it would be advantageous to use an unbiased approach that facilitates potential discoveries of new mechanisms of action that govern the pro-regenerative effect of SKM.

Overall, the data supports the hypothesis that SKM hydrogel is a promising therapeutic for the treatment of fibrosis in the tongue. In this rat model of tongue fibrosis, significant histomorphological improvements following SKM hydrogel injection were demonstrated. Gene expression data further suggest immunomodulation towards a pro-regenerative phenotype and improvement of angiogenesis, which is important for vascularization of the damaged tissues. This study encourages further investigation of SKM for the regeneration of damaged tongue tissue for the potential treatment of dysphagia.

### Example 2: SKM Hydrogel Formulation

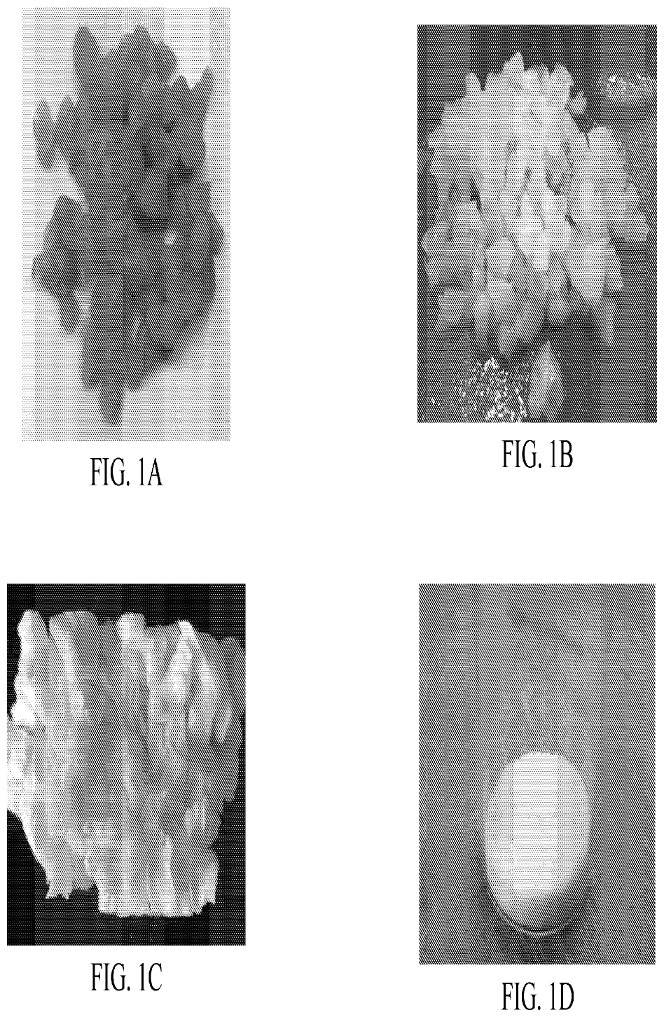

Skeletal muscle matrix material was derived through decellularization of porcine skeletal muscle tissue.²⁰ Fat and connective tissue was removed, and the skeletal muscle was cut into ˜1 cm3 pieces (FIG. 1A), rinsed with deionized water and stirred in 1% (wt/vol) solution of sodium dodecyl sulfate (SDS) detergent for 5 days, with daily solution changes. The decellularized muscle (FIG. 1B) was then stirred overnight in deionized water, and agitated rinses under running deionized water were performed to remove residual SDS. In addition to confirmation with lack of nuclei on H&E stained sections (not shown), the DNA content of the material was measured as 26.14±1.67 ng of DNA/mg of dry weight ECM, which confirmed decellularization. The matrix was then frozen at −80° C., lyophilized for 48 hours (FIG. 1C), and milled into a fine particulate (FIG. 1D). At this stage the material was enzymatically digested with pepsin for 48 hours to form a liquid (FIG. 1E). The pH of the liquid ECM was neutralized and the ionic concentration was balanced, bringing the ECM to physiological conditions.¹⁸ A hydrogel (FIG. 1F) was thereafter formed at a concentration of 6 mg/mL, which was determined as the optimal concentration for skeletal muscle injection.²¹

### Example 3: Partial Glossectomy Model

The partial glossectomy model disclosed herein was developed to assess the efficacy of SKM hydrogel injection in reducing fibrosis and improve skeletal muscle regeneration.^{16,17,18} Procedures for developing the partial glossectomy model disclosed herein were approved by the Institutional Animal Care and Use Committee at the University of California, San Diego.

A partial glossectomy model was developed by inducing a partial glossectomy injury in male Sprague Dawley rats weighing between 225-250 g (approximately 3 months old).⁹ Briefly, animals were anesthetized with isoflurane, and buprenorphine was delivered subcutaneously for pain management. The tongue was retracted with a 4-0 silk suture. A 4-mm dermal punch was used to excise a quarter of the tongue, anterior to the circumvallate papillae on the left side. Silver nitrate chemical cautery was used for hemostasis. The animals were then monitored for 5 days postoperatively and were provided with a soft diet for the duration of the study period.

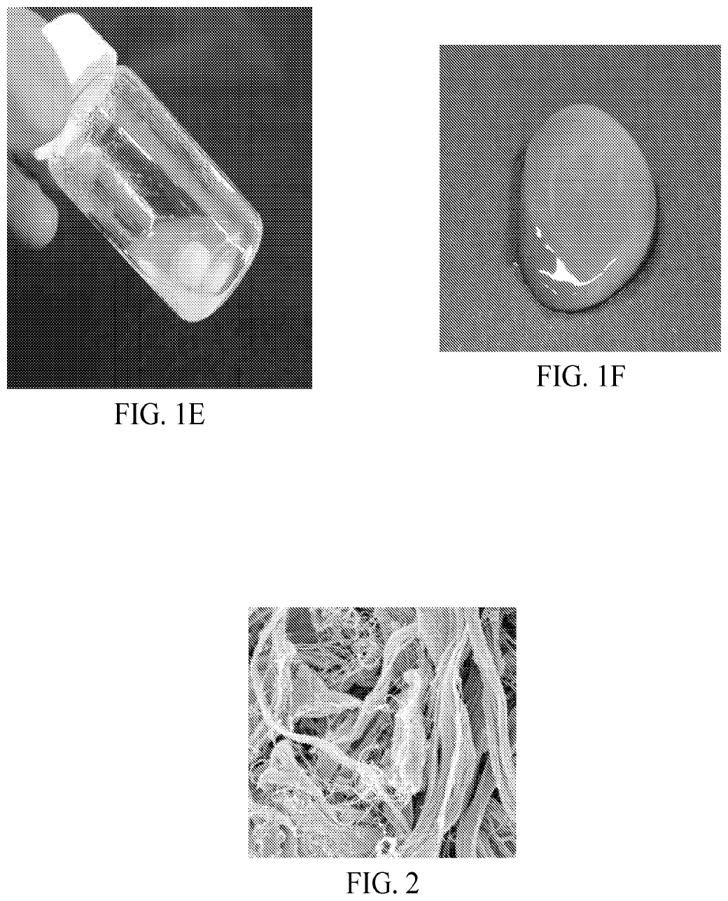

Scar formation at the site of injury occurred over the following 2 weeks,⁹ at which time point SKM hydrogel was injected directly into the tongue. FIG. 2 depicts the SKM hydrogel 20 minutes post-injection. Because acellular biomaterial therapeutics have not been previously investigated for oropharyngeal dysphagia in a pre-clinical animal model, a study was necessary to determine the optimal injection volume of SKM hydrogel. The study design included injection volumes ranging from 50 μL to 300 μL. Specifically, injection volumes of 50 μL, 100 μL, 200 μL, and 300 μL were assessed.

### Example 4: Reliability of the SKM Hydrogel Injection and Volume Optimization

To determine the optimal injection volume for effectively treating a partial glossectomy injury with SKM hydrogel, injection volumes of 50 μL, 100 μL, 200 μL, and 300 μL were tested (n=2 animals per volume). This range of injection volumes was determined based on SKM injections in other skeletal muscle injury models²¹ and cell injections in the rat tongue.⁹ The SKM hydrogel, at the various injection volumes, was injected directly into the site of injury (e.g., fibrotic scar), two weeks following a partial glossectomy injury.

The SKM hydrogel was pre-labeled with Alexa Fluor™ 568 NHS Ester (Thermo Fisher Scientific, Waltham, MA) to enable visualization of the SKM hydrogel in situ, the SKM hydrogel was prelabeled with Alexa Fluor™ 568 NHS Ester (Thermo Fisher Scientific, Waltham, MA). Fluorescent pre-labeling of the SKM hydrogel was achieved by incubating the SKM hydrogel with the Alexa Fluor™ 568 NHS Ester dye on ice for an hour to ensure complete binding.

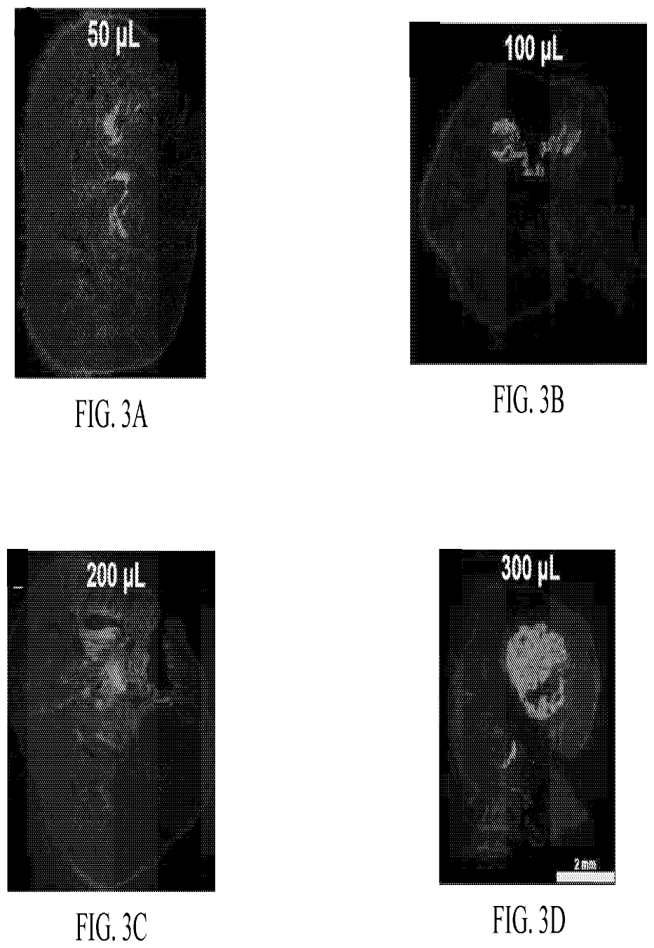

One week following the injection of SKM hydrogel, the animals were euthanized. The animals' tongues were harvested, cross-sectioned, and stained with DAPI nuclear counterstain for tissue localization. As shown in FIGS. 3A-3D, the tissue cross-sections were stained and visualized and 20× magnification using a Leica Ariol® fluorescent microscope. The representative fluorescent images of FIGS. 3A-3D show pre-labeled SKM hydrogel in red against a DAPI counterstain in blue to indicate cell nuclei.

Based on previous ECM hydrogel studies, greater material retention led to improved repair with a typical degradation time of approximately 3 weeks. It was therefore desirable to see high material retention in the tissue at 1 week post-injection to enable prolonged pro-regenerative cues and sufficient repair.^{18,21} As shown in FIGS. 3A and 3B, representing injection boluses of 50 μL and 100 μL, respectively, retention of the red pre-labeled SKM hydrogel injection was relatively low after 1 week. In contrast, retention of the 200 μL and 300 μL boluses, shown in FIGS. 3C-3D, respectively, was much higher. Because the representative images of injection boluses shown in FIGS. 3A-3D demonstrate increased material retention and spread through the tongue with higher injection volumes, 200 μL and 300 μL injection volumes were suitable for further investigation.

### Example 5: Histomorphological Assessment of SKM Therapeutic Efficacy

To study histomorphological changes following SKM hydrogel injection, SKM hydrogel or saline was injected into the injury site two weeks following partial glossectomy injury. Because the 200 μL and 300 μL injection volumes demonstrated good retention and spread in the tongue tissue, experimental groups included 200 μL SKM hydrogel, 300 μL SKM hydrogel, and 200 μL saline (n=6/group). This sample size was based on a previous study of SKM hydrogel for muscle regeneration in a model of hindlimb ischemia.⁵ Because the initial SKM hydrogel dosing study was a pilot study that did not aim to achieve statistically significant results, n=2 animals per injection volume were used for the initial investigation.

Using G*Power, 6 animals/group were needed to achieve 90% power and a significance of 0.05. For gene expression studies, preliminary data with qRT-PCR were used to calculate the sample size, and 11 animals/group/time point were necessary to achieve 80% power and a significance of 0.05. Data that followed a parametric distribution were compared using a t-test, or a one-way analysis of variance followed by Tukey's post hoc pairwise comparisons. Data that did not follow a parametric distribution (skeletal muscle cross-sectional fiber area) were analyzed by Mann-Whitney test.¹⁹ Data were analyzed using GraphPad Prism v8.0, San Diego, CA. Gene expression normalization and differential expression was analyzed using the NanoStringDiff package in R, with a significance at a p<0.05.19.

To investigate therapeutic potential of SKM hydrogel, the chosen injection volumes of 200 μL and 300 μL were injected 2 weeks following partial glossectomy injury. The experimental control group received a 200 μL saline injection at the same time point. 200 μL of saline was selected as the control at least in part because the 300 μL SKM hydrogel injection was initially anticipated to be inferior to 200 μL injection. In particular, the 300 μL was anticipated to be inferior because it was believed that the larger injection volume would be disruptive to the tissue.

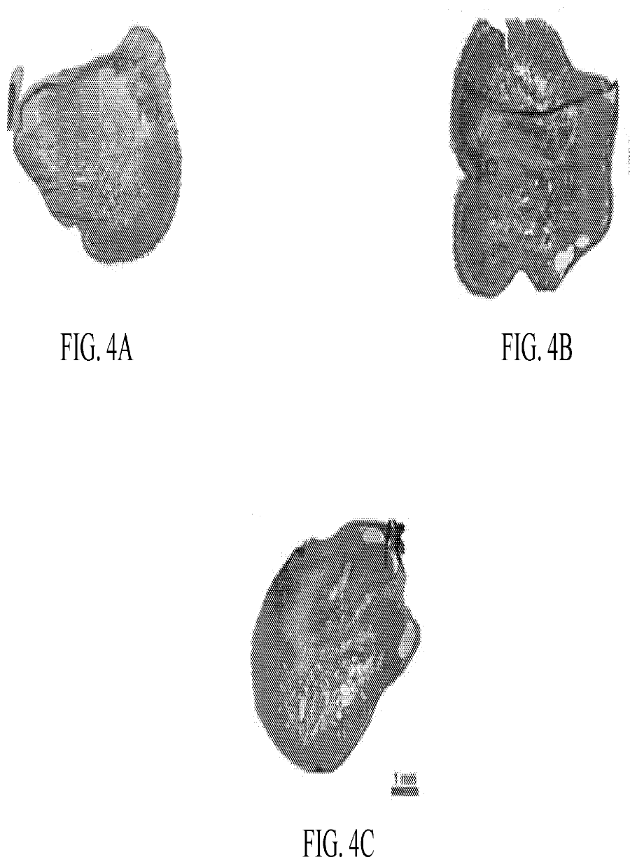

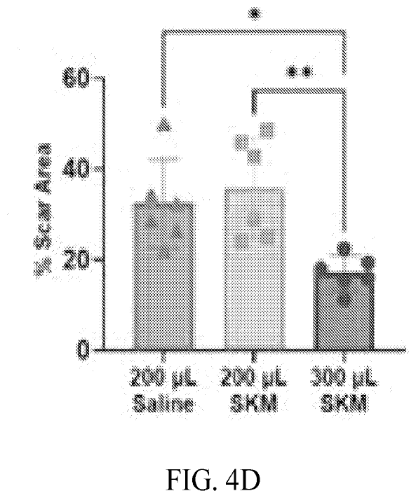

One week following the injection of SKM hydrogel or saline, the animals were euthanized. The animals' tongues were harvested, cross-sectioned and stained with Masson's Trichrome (Polysciences, Warrington, PA) stain. This stain was used to identify collagen and muscle within the tissue cross-sections. To quantify muscle fibers within the scar area, fibrosis was identified with an anti-collagen antibody (Bio-Rad, Hercules, CA, 1:500) with an Alexa Fluor™ 488 secondary (Invitrogen, Carlsbad, California, 1:500) and myofiber membranes were identified with anti-α-sarcoglycan antibody (Leica Biosystems, Wetzlar, Germany, 1:200) with an Alexa Fluor™ 568 secondary (Invitrogen, Carlsbad, California, 1:500). Tissue sections were visualized using a Leica Ariol® ScanScope® CS2 fluorescent microscope. As shown in FIGS. 4A-4C, muscle fibers appear red and collagen, denoting the scar region, appears green under the fluorescent microscope, and cellular nuclei appear blue.

The tongues were harvested 4 weeks after injection for assessment of histomorphological properties. To quantify the scar area from the Masson's Trichrome stained tissue sections, Aperio ImageScope software was used to trace the border of the scar region, as defined by the blue collagen stain. The outer edge of the tongue cross-section was traced, and the area of the scar region was normalized to total tongue area for each section. All sections containing the scar were analyzed, and normalized scar area data were averaged per animal. For quantification of muscle fibers from immunohistochemical staining, the border of the scar was identified with an anti-collagen antibody. Then, cross-sectional muscle fibers within the scar region were identified with the anti-α-sarcoglycan antibody and fiber areas were quantified. Muscle fibers with centralized nuclei (identified with DAPI stain) were also identified. Numbers of fibers were normalized to scar area, and the numbers of centrally nucleated fibers were normalized to muscle fiber counts. All sections containing scar were analyzed, and data were averaged per animal.

Representative Brightfield images of tongue cross-sections from the treatment groups are shown in FIGS. 4A-4C. In particular, FIG. 4A depicts an image of the 200 μL saline injection, FIG. 4B depicts an image of the 200 μL SKM hydrogel injection, and FIG. 4C depicts an image of the 300 μL SKM hydrogel injection. It was observed that SKM hydrogel injection is effective to decrease scar formation in this injury model, which is important as fibrosis contributes to dysphagia following surgical resection and radiation of head and neck cancers. In particular, both the 200 μL injection and the 300 μL injection of the SKM hydrogel was shown to be well-retained and well-dispersed in the scar regions of the tissue samples. Treatment groups were compared using one-way ANOVA with Tukey multiple comparisons test (*p<0.05; **p<0.01), where the fibrotic scar area was normalized to the tissue area per section for the purpose of comparison. The comparison of treatment groups indicated that scar area fraction was significantly reduced in the 300 μL SKM group compared to both 200 μL SKM (P=0.005) and 200 μL saline (P=0.02) groups, as shown in FIG. 4D.

To more precisely assess the impact of SKM hydrogel injection on muscle fiber formation, reduction of scar formation was further assessed in samples from the 300 μL SKM hydrogel injection group and the saline group. Samples from the 200 μL SKM hydrogel injection group were excluded from this analysis, as those samples failed to demonstrate a significant reduction in scar formation.

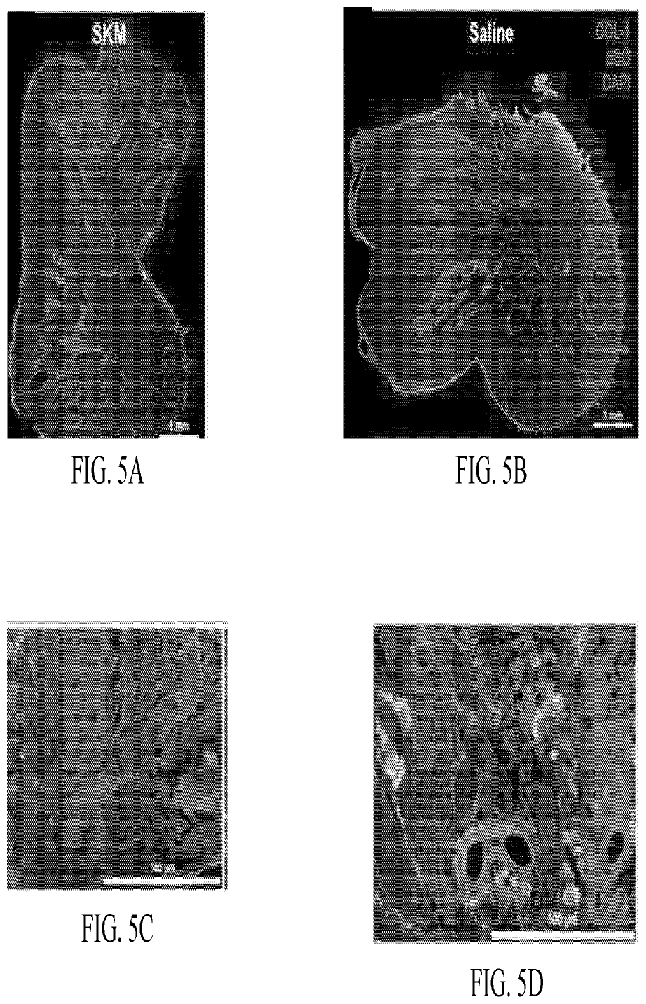

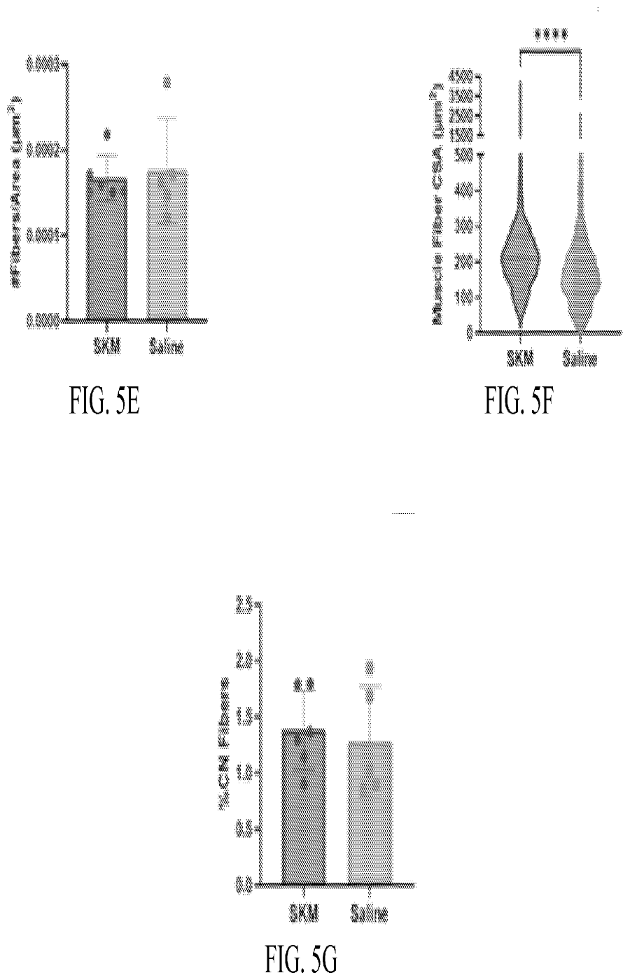

Short axis cross-sections of tongue specimens were stained with antibodies against collagen I and myofibers membranes (α-sarcoglycan), and a nuclear stain (DAPI). Representative fluorescent images are shown in FIGS. 5A-5D. FIGS. 5A and 5C depict a low resolution and a high resolution image of the stained sample injected with 300 μL SKM hydrogel, respectively. FIGS. 5B and 5D depict a low resolution and a high resolution image of the stained sample injected with saline, respectively. The higher magnification images of FIGS. 5C and 5D more clearly depict differences in fiber size and organization within the region of scar. Moreover, as shown in FIG. 5C, the stained sample injected with 300 μL SKM hydrogel demonstrates the presence of skeletal muscle fibers inside the scar area.

To assess the effect of SKM hydrogel injection on muscle regeneration, the fiber number and cross-sectional area inside the scar region were quantified. Although no quantitative differences in the fiber number were observed between the groups (P=0.86), as shown in FIG. 5E, the fiber cross-sectional area was significantly greater in the 300 μL SKM hydrogel compared to saline group (p<0.0001), as shown in FIG. 5F. The percentage of centrally nucleated fibers was also quantified to determine if the muscle was actively regenerating, however there was no difference in this percentage between SKM hydrogel and saline treated animals, as shown in FIG. 5G, indicating that muscle regeneration was not ongoing and the fiber area measurements were reflective of the tissue at homeostasis.

### Example 6: RNA Isolation and Nanostring Multiplex Gene Expression Analysis

Having demonstrated significant histomorphological improvements with SKM hydrogel injection in this model, a gene expression study was conducted to investigate potential mechanisms driving these changes. Two weeks after partial glossectomy injury, either 300 μL of SKM hydrogel or Saline was injected into the injury site (n=6/group). The 300 μL SKM hydrogel injection volume was chosen based on histological data, and the control saline injection was increased to 300 μL to match SKM hydrogel volume. An injured non-injected group was used as an additional control to further evaluate potential effects of the injection itself. At 3 and 7 days post-injection, physiologically relevant timepoints for the immune response and early muscle regeneration, tongues were harvested and submerged in RNAlater™, then stored at 4° C. overnight before being transferred to −80° C. to preserve tissues for RNA isolation. For RNA isolation, tongue specimens were thawed and tissue was trimmed to isolate the scar region. Isolated scar region was divided in half, to accommodate spin column capacity, and homogenized (TissueRuptorII, Qiagen, Germantown, Maryland), and RNA was isolated with RNAeasy Fibrous Tissue Mini Kit following manufacturer instructions (Qiagen, Germantown, Maryland).

A NanoString nCounter® MAX Analysis System with an nCounter® custom CodeSet of 145 genes involved in pathways relevant to skeletal muscle regeneration.¹⁰ The pathways relevant to skeletal muscle regeneration include the immune response, myogenesis, muscle anabolism/catabolism, angiogenesis, and ECM remodeling, which were used to characterize the tissue response to SKM injection. RNA concentration was measured using a Qubit 3.0 Fluorometer with a Qubit™ RNA HS Assay kit. The hybridization buffer (70 μL) was then mixed with the Custom Reporter CodeSet solution, and 8 μL of this master mix was then added to 50-100 ng of RNA per tissue sample, and RNA-free water up to 13 μL total. Then, 2 μL of Capture ProbeSet was added to the mixture, thoroughly mixed and placed on a thermocycler at 65° C. for 16-48 hours and then maintained at 4° C. for less than 24 hours. Using a two-step magnetic beads purification, probe excess was removed in PrepStation and target/probe complexes were bound on the cartridge. The data were collected by the digital analyzer (NanoString nCounter® Digital Analyzer) with images of immobilized fluorescent reporters in the sample cartridge. Results of barcode reads were analyzed by nSolver™ Analysis Software 4.0, and differential expression analysis was done with a custom R script. The NanoString data were visualized using ggplot and pheatmap packages in R.

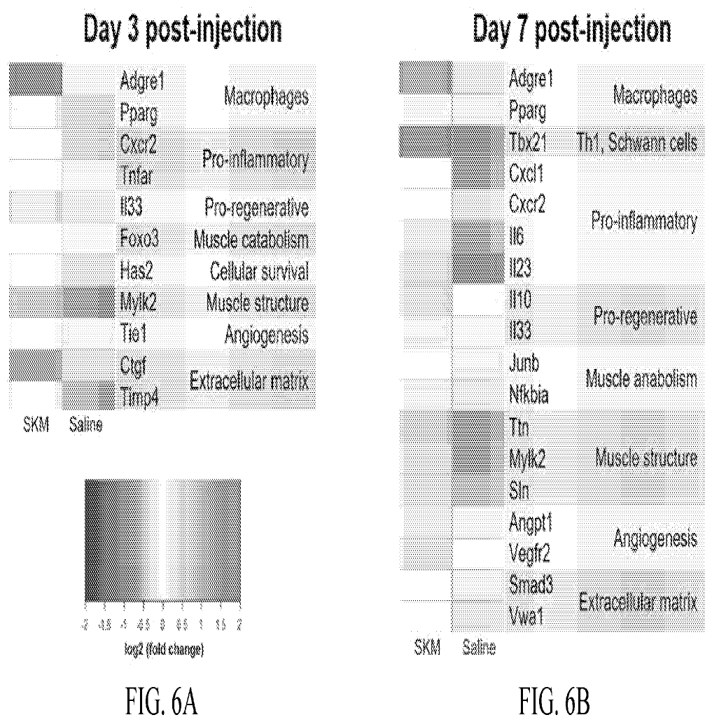

The significant differentially expressed genes between SKM hydrogel and saline injections are listed for days 3 and 7 post-injection alongside their associated pathways in FIGS. 6A and 6B, respectively. To visualize the relative differences in expression for each gene between SKM hydrogeland saline groups, heatmaps were generated using fold changes with respect to non-injected injured controls. At day 3 post-injection, SKM hydrogel led to upregulation of Adgre1, a macrophage marker, and Pparg, a macrophage transcriptional marker of pro-repair macrophages.²² SKM hydrogel immunomodulation was also observed through the downregulation of pro-inflammatory chemokine Ccr2 and upregulation of pro-regenerative cytokine Il33 with respect to saline. SKM hydrogel injection upregulated expression of Has2, a hyaluronic acid synthase, and Mylk2, involved in skeletal muscle contraction, compared to saline injection. SKM hydrogel injection also resulted in upregulation of Ctgf, which is known for a pro-reparative role in wound healing when expressed transiently in early injury response.²³ Compared to saline, the SKM hydrogel group had higher expression of Timp4, a matrix metalloproteinase inhibitor that regulates ECM remodeling as well as angiogenesis.²⁴ Tie1, another gene involved in angiogenesis, was also upregulated in SKM with respect to saline.

At day 7 post-injection, SKM hydrogel upregulation of macrophage markers was sustained (Adgre1), and upregulation of Tbx21, a marker for type 1 T-helper cells, was also observed. Chemokines (Cxcl1, Cxcl2) and cytokines (IL6, IL23) associated with a pro-inflammatory response were downregulated in the SKM hydrogel group compared to saline, while cytokines associated with an anti-inflammatory or pro-regenerative response (IL10, IL33) were upregulated. While SKM hydrogel injection did not upregulate genes related to muscle anabolism (Junb, Nfkbia), an upregulation of genes associated to muscle structure (Ttn, Mylk2, Sln) was observed in the SKM hydrogel group, as compared to saline. Finally, it was observed that SKM hydrogel injection induced upregulation of angiogenesis genes Angpt1 and Vegfr2. Overall, the gene expression data from both day 3 and 7 post-injection are consistent in demonstrating that SKM hydrogel injection reduces pro-inflammatory response, increases pro-regenerative response, and increases angiogenic signaling.

TABLE 1
Differentially expressed genes between
SKM and Saline at 3 days post-injection

| Gene ID | logFC | pvalue |
|---|---|---|
| Adgr1 | 1.61850664 | 4.8004E−07 |
| Ctgf | 0.70633627 | 0.00414928 |
| Cxcr2 | −0.34528434 | 0.02096618 |
| Foxo3 | −0.12315037 | 0.04937981 |
| Has2 | 0.3513814 | 0.0205203 |
| Il33 | 0.27797367 | 0.00260988 |
| Mylk2 | 0.32294349 | 0.04255447 |
| Pparg | 0.38672879 | 0.00212215 |
| Tie1 | 0.19614778 | 0.00181974 |
| Timp4 | 0.53425493 | 0.00122851 |
| Tnfar | −0.1528032 | 0.02158274 |

TABLE 2
Differentially expressed genes between
SKM and Saline at 7 days post-injection

| Gene ID | logFC | pvalue |
|---|---|---|
| Angpt1 | 0.14253229 | 0.0229001 |
| Adgr1 | 1.12361604 | 4.9195E−10 |
| Il10 | 0.39355048 | 0.01879268 |
| Il23 | −1.05792432 | 0.00867724 |
| Il33 | 0.28718578 | 0.00116824 |
| Il6 | −0.70274986 | 0.04404408 |
| Junb | −0.23130409 | 0.03881962 |
| Mylk2 | 0.43166396 | 0.01019355 |
| Nfkbia | −0.39847947 | 0.00092809 |
| Pparg | 0.24223418 | 0.00903286 |
| Sln | 0.25567115 | 0.04625474 |
| Smad3 | 0.21059108 | 0.00569874 |
| Ttn | 0.35984638 | 0.04332277 |
| Tbx21 | 0.84771311 | 0.01019494 |
| Vegfr2 | 0.30472051 | 0.01561083 |
| Vwa1 | −0.21981367 | 0.04637678 |
| Cxcl1 | −0.98049511 | 0.0487632 |
| Cxcr2 | −0.33182052 | 0.02507791 |

### REFERENCES

1. Dellis S, Papadopoulou S, Krikonis K, Zigras F. Sarcopenic Dysphagia. A Narrative Review. J Frailty, Sarcopenia Falls. 2018; 03(01):1-7. doi:10.22540/JFSF-03-001
2. Pauloski B R. Rehabilitation of Dysphagia Following Head and Neck Cancer. doi:10.1016/j.pmr.2008.05.010
3. Head and Neck Cancer: Statistics|Cancer.Net. Accessed Apr. 18, 2022. https://www.cancer.net/cancer-types/head-and-neck-cancer/statistics
4. Garcia-Peris P, Parón L, Velasco C, et al. Long-term prevalence of oropharyngeal dysphagia in head and neck cancer patients: Impact on quality of life. Clin Nutr. 2007; 26(6):710-717. doi:10.1016/J.CLNU.2007.08.006
5. Nguyen N P, Moltz C C, Frank C, et al. Dysphagia following chemoradiation for locally advanced head and neck cancer. Ann Oncol. 2004; 15(3):383-388. doi:10.1093/ANNONC/MDH101
6. Hyo Choi K, Ri Son Y, Gyun Kim T. Dysphagia in Tongue Cancer Patients. Ann Rehabil Med Orig Artic Ann Rehabil Med. 2015; 39(2):210-217. doi:10.5535/arm.2015.39.2.210
7. Langmore S E, Pisegna J M. Efficacy of exercises to rehabilitate dysphagia: A critique of the literature. Int J Speech Lang Pathol. 2015; 17(3):222-229. doi: 10.3109/17549507.2015.1024171
8. Tran E K, Juarez K O, Long J L. Stem cell treatments for oropharyngeal dysphagia: Rationale, benefits, and challenges. World J Stem Cells. 2020; 12(9):1001-1012. doi:10.4252/wjsc.v12.i9.1001
9. Vahabzadeh-Hagh A M, Goel A N, Frederick J W, Berke G S, Long J L. Transplanted Human Multipotent Stromal Cells Reduce Acute Tongue Fibrosis in Rats. Published online 2018. doi:10.1002/lio2.202
10. Nativ-Zeltzer N, Kuhn M A, Evangelista L, et al. Autologous Muscle-Derived Cell Therapy for Swallowing Impairment in Patients Following Treatment for Head and Neck Cancer. Published online 2021. doi: 10.1002/lary.29606
11. Spang M T, Christman K L. Extracellular matrix hydrogel therapies: In vivo applications and development. Acta Biomater. 2018; 68:1-14. doi:10.1016/j.actbio.2017.12.019
12. Rao N, Agmon G, Tierney M T, et al. Engineering an Injectable Muscle-Specific Microenvironment for Improved Cell Delivery Using a Nanofibrous Extracellular Matrix Hydrogel. ACS Nano. 2017; 11(4):3851-3859. doi:10.1021/acsnano.7b00093
13. Seif-Naraghi S B, Singelyn J M, Salvatore M A, et al. Safety and Efficacy of an Injectable Extracellular Matrix Hydrogel for Treating Myocardial Infarction. Sci Trans/Med. 2013; 5(173):173ra25 LP-173ra25. doi:10.1126/scitranslmed.3005503
14. Nezhad Z M, Poncelet A, De Kerchove L, Gianello P, Fervaille C, Khoury G El. Small intestinal submucosa extracellular matrix (CorMatrix®) in cardiovascular surgery: a systematic review. ZMosala Nezhad). Published online 2016. doi:10.1093/icvts/ivw020
15. Sarrafian T L, Bodine S C, Murphy B, Grayson J K, Stover S M. Extracellular matrix scaffolds for treatment of large volume muscle injuries: A review. Vet Surg. 2018; 47(4):524-535. doi:10.1111/VSU.12787
16. Traverse J H, Henry T D, Dib N, et al. First-in-Man Study of a Cardiac Extracellular Matrix Hydrogel in Early and Late Myocardial Infarction Patients. JACC Basic to Transl Sci. 2019; 4(6):659-669. doi:10.1016/j.jacbts.2019.07.012
17. Ungerleider J L, Johnson T D, Hernandez M J, et al. Extracellular Matrix Hydrogel Promotes Tissue Remodeling, Arteriogenesis, and Perfusion in a Rat Hindlimb Ischemia Model. JACC Basic to Transl Sci. 2016; 1(1-2):32-44. doi:10.1016/j.jacbts.2016.01.009
18. DeQuach J A, Lin J E, Cam C, et al. Injectable skeletal muscle matrix hydrogel promotes neovascularization and muscle cell infiltration in a hindlimb ischemia model. Eur Cells Mater. 2013; 23(1):400-412. doi:10.1161/CIRCULATIONAHA.110.956839
19. Duran P, Sesillo F B, Burnett L, et al. Pro-regenerative Extracellular Matrix Hydrogel Prevents and Mitigates Pathological Alterations of Pelvic Muscles Following Birth Injury. bioRxiv. Published online May 28, 2021:2021.05.28.446170. doi:10.1101/2021.05.28.446170
20. Ungerleider J L, Johnson T D, Rao N, Christman K L. Fabrication and characterization of injectable hydrogels derived from decellularized skeletal and cardiac muscle. Methods. 2015; 84:53-59. doi:10.1016/j.ymeth.2015.03.024
21. Hernandez M J, Zelus E I, Spang M T, Braden R L, Christman K L. Dose optimization of decellularized skeletal muscle extracellular matrix hydrogels for improving perfusion and subsequent validation in an aged hindlimb ischemia model. Biomater Sci. 2020; 8(12):3511-3521. doi:10.1039/c9bm01963d
22. Varga T, Mounier R, Patsalos A, et al. Macrophage PPAR&gamma, a Lipid Activated Transcription Factor Controls the Growth Factor GDF3 and Skeletal Muscle Regeneration. Published online 2016. doi:10.1016/j.immuni.2016.10.016
23. Alfaro M P, Deskins D L, Wallus M, et al. A physiological role for connective tissue growth factor in early wound healing. Lab Investig. 2012; 93:81-95. doi:10.1038/labinvest.2012.162
24. Alameddine H S, Morgan J E. Matrix Metalloproteinases and Tissue Inhibitor of Metalloproteinases in Inflammation and Fibrosis of Skeletal Muscles. J Neuromuscul Dis. 2016; 3:455-473. doi:10.3233/JND-160183
25. Hardy D, Besnard A, Latil M, et al. Comparative Study of Injury Models for Studying Muscle Regeneration in Mice. Published online 2016. doi:10.1371/journal.pone.0147198
26. Diaz M D, Tran E, Spang M, et al. Injectable Myocardial Matrix Hydrogel Mitigates Negative Left Ventricular Remodeling in a Chronic Myocardial Infarction Model. JACC Basic to Transl Sci. Published online 2021:1-12. doi:10.1016/j.jacbts.2021.01.003
27. Walker C L. Progress in perisynaptic Schwann cell and neuromuscular junction research. Neural Regen Res. 2022; 17(6):1273-1274. doi:10.4103/1673-5374.327334
28. Coletti D, Mccarthy J J, Scicchitano B M, et al. Fibro-Adipogenic Progenitors Cross-Talk in Skeletal Muscle: The Social Network. Published online 2019. doi:10.3389/fphys.2019.01074
29. Yang W, Hu P. Skeletal muscle regeneration is modulated by inflammation. J Orthop Transl. 2018; 13:25-32. doi:10.1016/j.jot.2018.01.002
30. Calve S, Isaac J, Gumucio J P, Mendias C L. Hyaluronic acid, HAS1, and HAS2 are significantly upregulated during muscle hypertrophy. Am J Physiol—Cell Physiol. 2012; 303(5):577-588. doi:10.1152/ajpcell.00057.2012
31. Aurora A, Roe J L, Corona B T, Walters T J. An acellular biologic scaffold does not regenerate appreciable de novo muscle tissue in rat models of volumetric muscle loss injury. Biomaterials. 2015; 67:393-407. doi:10.1016/j.biomaterials.2015.07.040
32. Nuutila K, Sakthivel D, Kruse C, Tran P, Giatsidis G, Sinha I. Gene expression profiling of skeletal muscle after volumetric muscle loss. doi:10.1111/wrr.12547
33. Birbrair A, Zhang T, Wang Z M, Messi M L, Mintz A, Delbono O. Type-I pericytes participate in fibrous tissue deposition in aged skeletal muscle. Am J Physiol—Cell Physiol. 2013; 305(11). doi:10.1152/ajpcell.00171.2013

1. A method of treating head and/or neck muscle injury or muscle in a subject, the method comprising administering to a subject in need an effective amount of an extracellular matrix (ECM) composition.

2. The method of claim 1, wherein the ECM composition is administered as a liquid.

3. The method of claim 1, wherein the ECM composition transforms to a gel form after being administered.

4. The method of claim 1, wherein the ECM composition is administered as a powder.

5. The method of claim 1, wherein the ECM composition is derived from skeletal muscle.

6. The method of claim 5, wherein the ECM composition comprises a skeletal muscle extracellular matrix (SKM) hydrogel.

7. The method of claim 1, wherein the ECM composition further comprises cells, drugs, proteins, or polysaccharides.

8. The method of claim 1, wherein the ECM composition is injected at the site of the head and/or neck muscle injury or muscle loss.

9. The method of claim 8, wherein the ECM composition is configured to degrade within three months following injection.

10. The method of claim 8, wherein the ECM composition is configured to degrade within one month following injection.

11. The method of claim 1, wherein the effective amount of the ECM composition comprises a concentration of about 1 mg to about 20 mg of ECM per mL of the ECM composition.

12. The method of claim 1, wherein the head and/or neck muscle injury or muscle loss is weakness or asymmetry in at least one facial muscle, tongue weakness, tongue volume loss, pharyngeal musculature weakness, laryngeal musculature weakness and/or muscle loss or thinning, bulking of the inferior turbinates within the nasal cavity, augmentation around the Eustachian tube for Eustachian tube dysfunction and patulous Eustachian tube, dysphagia, dysarthria, palate or pharyngeal incompetence, velopharyngeal insufficiency, tongue atrophy, scarring, or fibrosis formation.

13. The method of claim 12, wherein the head and/or neck muscle injury or muscle loss is caused by an oral cancer, oropharyngeal cancer, hypopharyngeal cancer, laryngeal cancer, cancer of the upper aerodigestive tract, or caused by a different treatment for said cancers.

14. The method of claim 1, wherein administering the effective amount of the ECM composition is effective to promote muscle regeneration, engraftment, growth, volume, bulk, or function.

15. A method of using an extracellular matrix (ECM) composition, the method comprising:
administering an effective amount of the ECM composition to a subject in need;
wherein administering the ECM composition is effective to treat head and/or neck muscle injury or muscle loss in the subject.

16. The method of claim 15, wherein the ECM composition comprises a skeletal muscle extracellular matrix (SKM) hydrogel.

17. The method of claim 15, wherein the ECM composition is injected at the site of the head and/or neck muscle injury or muscle loss, infused via a catheter, administered intravenously, or administered via intravascular infusion.

18. The method of claim 15, wherein the effective amount of the ECM composition comprises a concentration of about 1 mg to about 20 mg of ECM per mL of the ECM composition.

19. The method of claim 15, wherein the effective amount of the ECM composition is administered to the subject once.

20. The method of claim 15, wherein the effective amount of the ECM composition is administered to the subject periodically at an interval of once per day, once per week, once per month, or once per year.

21. The method of claim 15, wherein the head and/or neck muscle injury or muscle loss is caused by an oral cancer, oropharyngeal cancer, hypopharyngeal cancer, laryngeal cancer, cancer of the upper aerodigestive tract, or caused by a different treatment for said cancers, and wherein administering the effective amount of the ECM composition is effective to promote muscle regeneration, engraftment, growth, volume, bulk, or function.

22. A method of formulating an extracellular matrix (ECM) composition, the method comprising:
isolating a tissue sample;
decellularizing the tissue sample;
lyophilizing the tissue sample;
milling the tissue sample; and
enzymatically digesting the tissue sample;
wherein the ECM composition is effective to treat head and/or neck muscle injury in a subject.

23. The method of claim 22, wherein the tissue sample comprises skeletal muscle.

24. The method of claim 23, wherein the extracellular matrix (ECM) composition comprises a skeletal muscle extracellular matrix (SKM) hydrogel effective to promote muscle regeneration, engraftment, growth, volume, bulk, or function.

25. The method of claim 22, wherein the head and/or neck muscle injury or muscle loss is caused by an oral cancer, oropharyngeal cancer, hypopharyngeal cancer, laryngeal cancer, cancer of the aerodigestive tract, or caused by a different treatment for said cancers, such that the head and/or neck muscle injury is weakness or asymmetry in at least one facial muscle, tongue weakness, pharyngeal musculature, laryngeal musculature weakness and/or thinning, bulking of the inferior turbinates within the nasal cavity, augmentation around the Eustachian tube for Eustachian tube dysfunction and patulous Eustachian tube, dysphagia, dysarthria, palate or pharyngeal incompetence, velopharyngeal insufficiency, tongue atrophy, scarring, or fibrosis formation.

26. The method of claim 22, wherein the effective amount of the ECM composition comprises a concentration of about 1 mg to about 20 mg of ECM per mL of the ECM composition, and wherein the effective amount of the extracellular matrix (ECM) composition comprises a skeletal muscle extracellular matrix (SKM) hydrogel effective to promote muscle regeneration, engraftment, growth, volume, bulk, or function.
